(19) United States
(12) Patent Application Publication
(10) Pub. No.: US 2014/0154337 A1
(43) Pub. Date: Jun. 5, 2014

(54) ANTIMICROBIAL AGENTS AND METHODS FOR USE THEREOF IN THE ABATEMENT OF FOOD BORNE PATHOGENIC ORGANISMS

(71) Applicant: Lynn C. Wilford, Grand Prairie, TX (US)

(72) Inventor: Lynn C. Wilford, Grand Prairie, TX (US)

(21) Appl. No.: 14/173,527

(22) Filed: Feb. 5, 2014

Related U.S. Application Data
(63) Continuation of application No. PCT/US12/50153, filed on Aug. 9, 2012.
(60) Provisional application No. 61/521,469, filed on Aug. 9, 2011.

(51) Int. Cl. A61K 33/32; A61K 31/194; A61K 33/22; A61K 33/06; A61K 33/30; A61K 31/538; A61K 33/26

(52) U.S. Cl. CPC A61K 33/32; A61K 31/538; A61K 31/194; A61K 33/26; A61K 33/06; A61K 33/30; A61K 33/22
USPC 424/639; 424/646; 424/682; 424/641; 424/657; 424/642

(57) ABSTRACT

Antimicrobial agents and methods for the abatement of food borne pathogenic organisms—particularly, Escherichia coli—include the steps of providing a specifically formulated humic composition and, thereafter, applying the humic composition as an antimicrobial agent for the abatement of Escherichia coli. In at least some applications of the present invention, other substances are introduced in combination with the humic composition thereby synergistically providing in certain applications a greater effect than otherwise independently obtainable.

### RELATED APPLICATIONS

This application claims priority to and all available benefit of P.C.T. international application No. PCT/US12/50153 filed Aug. 9, 2012 and U.S. provisional patent application Ser. No. 61/521,469 filed Aug. 9, 2011. By this reference, the full disclosures of P.C.T. international application No. PCT/US12/50153 and U.S. provisional patent application Ser. No. 61/521,469 are incorporated herein as though now set forth in their respective entireties.

### FIELD OF THE INVENTION

The present invention relates to food safety. More particularly, the present invention relates to a humic composition adapted for use as a topical or enteral antimicrobial and, in particular, as such a microbial having efficacy in the abatement of food borne pathogenic organisms—particularly, Escherichia coli.

### BACKGROUND OF THE INVENTION

The presence in and about the food chain of food borne pathogenic and/or otherwise harmful organisms is a persistent problem presenting the food industry enormous costs in terms of both financial resources and reputation. Worse, however, the presence of such organisms inflicts even greater costs in terms of sickness and death. In particular, such organisms as Escherichia coli, most particularly, but also Streptococcus, Staphylococcus, Listeria, Campylobacter, Salmonella and the like, afflict their harm on countless people on a daily basis.

With the costs so high, methods for control of these dangerous pathogens are desperately needed. As a result, it is an overriding object of the present invention to improve generally over the prior art by setting forth safe and reliable formulation and methods for the use thereof such that the incidence of sickness and death associated with at least some food borne pathogenic organisms may finally be curtailed. Additionally, it is an object of the present invention to set forth such methods and formulations that may be aggressively introduced over the widest spectrum of the food chain, thereby ensuring as impactful as possible result. Finally, it is an object of the present invention to set forth such methods and formulations that are economical in implementation, thereby ensuring that the benefits provided may be had by the greatest possible number of persons.

### SUMMARY OF THE INVENTION

In accordance with the foregoing objects, the present invention—a method for use of a novel antimicrobial agent in the abatement of food borne pathogenic organisms—generally comprises the steps of providing a humic composition (as herein expressly defined) and, thereafter, applying the humic composition as an antimicrobial agent for the abatement of Escherichia coli. In at least some applications of the present invention, other substances are introduced in combination with the humic composition, thereby synergistically providing in certain applications a greater effect than otherwise independently obtainable.

The humic composition (or combination thereof) is in a first set of uses epicutaneously or analogously applied as a topical antimicrobial to produce a local effect against surface Escherichia coli while in a second set of uses the humic composition (or combination thereof) is administered through an oral, nasal or like route to an animal, whereafter the humic composition (or combination thereof) acts as an antimicrobial to produce an effect against enteric or otherwise acting Escherichia coli.

Finally, other features, objects and advantages of the present invention will be apparent to those of ordinary skill in the relevant arts, especially in light of the foregoing discussions and the following exemplary detailed description and appended claims.

### DETAILED DESCRIPTION OF THE PREFERRED EMBODIMENT

Although those of ordinary skill in the art will readily recognize many alternative embodiments, especially in light of the illustrations provided herein, this detailed description is exemplary of the preferred embodiment of the present invention, the scope of which is limited only by the claims appended hereto.

Humic composition. As used herein, the term “humic composition” is expressly defined as a composition of matter generally comprising a subcombination of humic acid and fulvic acid in a ratio of 95-98% humic acid and 5-2% fulvic acid in combination with a quantity of one or more elements including at least one of iron, manganese, magnesium, zinc or boron.

The present invention generally comprises the steps of providing a humic composition (as herein expressly defined) and, thereafter, applying the humic composition as an antimicrobial agent for the abatement of Escherichia coli. Additionally, however, and as will be better understood further herein, in at least some applications of the present invention, other substances are introduced in further combination with the humic composition thereby synergistically providing in certain applications a greater effect than otherwise independently obtainable. As also will be better understood further herein, the humic composition (or combination formed thereof) is in a first set of uses epicutaneously or analogously applied as a topical antimicrobial to produce a local effect against surface borne Escherichia coli while in a second set of uses the humic composition (or combination formed thereof) is administered through an oral, nasal or like route to an animal, whereafter the humic composition (or combination formed thereof) acts as an antimicrobial to produce an effect against enteric or otherwise acting Escherichia coli.

### Topical Formulation Example 1

In a first example of a topical formulation according to the teachings of the present invention, the humic composition (as herein expressly defined) is placed in solution for use as a spray, wash or dip for epicutaneous or analogous application as a topical antimicrobial effective to kill, retard growth of or otherwise abate Escherichia coli, especially including E. coli 157. As will be appreciated by those of ordinary skill in the art, the solution may be created by simply mixing the humic composition with purified water. In any case, the topical formulation prepared for use as a spray or wash may then be applied to meat products during processing at slaughter houses, meat packing plants and/or other meat processing operations; applied to surfaces and/or equipment contacting meat products or otherwise utilized in proximity to meat products at slaughter houses, meat packing plants and/or other meat processing operations such as, for example, conveyor belts, cutters, saws, hooks, grinders, bins, tubs, cutting surfaces, knives, walkways and/or floors; applied during harvesting, processing, transporting or other handling to produce and especially to such produce as spinach, lettuce or other leafy greens, peanuts or other nuts, fruits and like produce that pose an elevated risk of contamination concomitant their exposure to soil, manure, dirt and/or sand such as often harbor Escherichia coli; applied to plant seeds, stems and the like as a pretreatment soak or the like prior to or during planting or, as an additive to irrigation water, at any stage of plant growth; applied as a disinfectant in hospitals or other healthcare facilities such as are generally challenged by the presence of infectious agents in general and Escherichia coli in particular in, on or about common areas, treatment rooms, operating theaters, preparation and/or recovery rooms, intensive care facilities and/or equipment; applied to surgical, breathing or like masks or to other surfaces as a prophylactic against airborne Escherichia coli; applied directly to a wound as a wound care treatment; applied in food service establishments such as, for example, restaurants, cafeterias and the like, to food handling surfaces and equipment such as, for example, countertops, cutting boards, knives, machinery and the like; applied as a surface disinfectant in and/or about livestock handling facilities including, in particular, milking parlors and the like; and/or injected into the water supply of a mister for delivery in the mist in control of airborne or dust-bound Escherichia coli.

### Laboratory Evaluation

In validation of Applicant's discoveries, controlled laboratory testing under Applicant's confidential direction confirms that the humic composition (as herein expressly defined) is effective as a topical antimicrobial against Escherichia coli. In particular, testing was conducted as follows:

1. Six (6) approximately identical lettuce leaves were inoculated with Escherichia coli of the American Type Culture Collection (“ATCC”) strain number 8739 (commonly referred to as the “Crooks strain”), which, as is known to those of ordinary skill in the art, is a feces derived strain typical of that expected in the applications of the present invention.

2. The inoculated samples were then randomly separated into two (2) groups of three (3) samples each, which groups were treated as follows:
a. Group I was sprayed with a solution of the humic composition in water; and
b. Group II was sprayed with purified water only (in order to establish a negative control for the evaluation).

3. After three days time, all samples were enumerated for the growth of Escherichia coli, wherein it was found that:
a. The samples of Group I showed no detectable growth of Escherichia coli, indicating a 100% kill rate; and
b. The samples of Group II showed the growth of Escherichia coli to maintain a clearly fatal level, indicating that under the experimental conditions of the test the Group I enumeration was significant in establishing efficacy of the application.

It is also noted that the laboratory testing as described also indicates that the application of the humic composition according to the teachings of the present invention very advantageously produces a residual efficacy over an extended period of up to several days time, which, of course, may be critical in an environment where re-exposure to pathogenic or otherwise harmful organisms may generally be expected or, likewise, in an environment otherwise conducive to re-colonization of a previously retarded or otherwise abated pathogenic or otherwise harmful organism.

### Topical Formulation Example 2

In a second example of a topical formulation according to the teachings of the present invention, the humic composition (as herein expressly defined) is combined with an organic oil and/or an organic acid and applied as a spray, wash and/or dip to the udders, teats, quarters, stomachs and/or legs of a dairy animal before and/or after milking. In this manner, a topical antimicrobial effective to kill, retard growth of or otherwise abate Escherichia coli, especially including E. coli 157, is obtained. Additionally, this combination is believed to be effective in the prevention and/or treatment of mastitis.

### Topical Formulation Example 3

In a third example of a topical formulation according to the teachings of the present invention, the humic composition (as herein expressly defined) is combined with an organic acid such as, for example, citric acid, lactic acid or boric acid for use as a topical antimicrobial effective to kill, retard growth of or otherwise abate Escherichia coli, especially including E. coli 157. In use, this combination is applied as a spray, wash or dip to the external surfaces of produce, where the combination may be expected to show residual efficacy over an extended period of up to several days time.

### Topical Formulation Example 4

In a fourth example of a topical formulation according to the teachings of the present invention, the humic composition (as herein expressly defined) is mixed with any of various personal care products as are otherwise topically applied such as, for example, cosmetics, lotions, soaps, shampoos and/or deodorants. In this manner, a topical antimicrobial effective to kill, retard growth of or otherwise abate Escherichia coli, especially including E. coli 157, is obtained in a form that encourages regular use without necessity for any action outside of the user's ordinary activities. As a result, this fourth topical formulation has the benefit of preventing infection even in the complete absence a priori of knowledge of a specific threat.

### Topical Formulation Example 5

In a fifth example of a topical formulation according to the teachings of the present invention, the humic composition (as herein expressly defined) is in dry form placed or otherwise deposited in a location where it may generally be expected that Escherichia coli will likely manifest such as, for example, in and about livestock handling facilities such as bed liners, livestock pens, stalls, cages, walkways, paths and/or feeding areas and/or milking parlors; in the airflow passage structures of air filters; and/or in the layer structures of activated carbon water filters.

### Enteral Formulation Example 1

In a first example of an enteral formulation according to the teachings of the present invention, the humic composition (as herein expressly defined) is administered orally to a mammal for use as an enteral antimicrobial effective to kill, retard growth of or otherwise abate naturally present enteric Escherichia coli, especially E. coli 157. In this formulation, the humic composition may simply be added to the ordinary dry or liquid foodstuff of the mammal and ingested therewith as is otherwise conventional.

### Laboratory Evaluation

In validation of Applicant's discoveries, controlled laboratory testing under Applicant's confidential direction confirms that the humic composition (as herein expressly defined) is effective as an enteral antimicrobial against Escherichia coli. In particular, testing was conducted as follows:

1. Six (6) identical conventional tryptic soy agar (“TSA”) plates were inoculated with Escherichia coli of the ATCC strain number 8739, which, as previously discussed, is a feces derived strain typical of that expected in the applications of the present invention.

2. The inoculated plates were then separated into three (3) groups of two (2) plates each, which groups were treated as follows:
a. Group I was sprayed with a mixture of the humic composition and 10% phosphoric acid solution in water, which phosphoric acid solution was added to obtain a pH of approximately 3.2 in simulation of the environment of a mammalian stomach;
b. Group II was sprayed with a mixture of 10% phosphoric acid solution in water in order to establish a control for the introduction of the phosphoric acid solution to the humic subcombination as applied to Group I; and
c. Group III was sprayed with purified water only (in order to establish a negative control for the evaluation).

3. After three days time, all plates were enumerated for the growth of Escherichia coli, wherein it was found that:
a. The plates of Group I showed no detectable growth of Escherichia coli, indicating a 100% kill rate;
b. The plates of Group II showed only a minor reduction in the growth of Escherichia coli to a level very near a lethal dosage, indicating that any effect of the phosphoric acid solution on the Group I enumeration was negligible; and
c. The plates of Group III showed an increase in growth of Escherichia coli to a clearly fatal level, indicating that under the experimental conditions of the test the Group I enumeration was significant in establishing efficacy of the application.

Due to the surprising nature of the obtained results, the entire test as described above was repeated. On repeat, nearly identical results were obtained to the same conclusions. It is also noted that the laboratory testing as described also indicates that the application of the humic composition according to the teachings of the present invention very advantageously produces a residual efficacy over an extended period of up to several days time, which, of course, may be critical in an environment where re-exposure to pathogenic or otherwise harmful organisms may generally be expected or, likewise, in an environment otherwise conducive to re-colonization of a previously retarded or otherwise abated pathogenic or otherwise harmful organism.

### Enteral Formulation Example 2

In a second example of an enteral formulation according to the teachings of the present invention, the humic composition (as herein expressly defined) is combined with a complex plant carbohydrate such as, for example, aloe and, thereafter, the combination is administered orally to a mammal for use as an enteral antimicrobial effective to kill, retard growth of or otherwise abate naturally present enteric Escherichia coli, especially E. coli 157. In the manner of the previously described enteral formulation, the combination of this formulation may simply be added to the ordinary dry or liquid foodstuff of the mammal and ingested therewith as is otherwise conventional. In addition to the benefits obtained with the previously described formulation, however, this second enteral formulation is also beneficial as a feed supplement. In particular, it is discovered that antioxidant properties of the complex plant carbohydrate serve to reduce inflammation in the udders of dairy animals, which in turn is believed to reduce susceptibility to mastitis while also dramatically increasing systemic absorption of the humic composition.

### Enteral Formulation Example 3

In a third example of an enteral formulation according to the teachings of the present invention, the humic composition (as herein expressly defined) is suspended in an otherwise conventional suppository and, thereafter, injected with the suppository into the rectum of a animal for use an enteral antimicrobial effective to kill, retard growth of or otherwise abate enteric Escherichia coli, especially E. coli 157.

While the foregoing description is exemplary of the preferred embodiment of the present invention, those of ordinary skill in the relevant arts will recognize the many variations, alterations, modifications, substitutions and the like as are readily possible, especially in light of this description. For example, Applicant now proposes that in light of the important discoveries as detailed herein, further study should be conducted to determine the efficacy of the described formulations and/or variants thereof utilized in the described methods and/or variants thereof for killing, retarding growth of or otherwise abating other pathogenic or otherwise harmful organisms including, but not limited to, Streptococcus, Staphylococcus, Listeria, Campylobacter, Salmonella and the like.

In any case, because the scope of the present invention is much broader than any particular embodiment, the foregoing detailed description should not be construed as a limitation of the scope of the present invention, which is limited only by the claims drawn hereto.

What is claimed is:

1. A method for abatement of Escherichia coli at a treatment location, said method for abatement comprising the steps of:
providing a humic composition, said humic composition comprising:
at least 95% humic acid;
at least 2% fulvic acid; and
a quantity of one or more elements including at least one of iron, manganese, magnesium, zinc or boron; and
delivering said humic composition as an antimicrobial agent to said treatment location.

2. The method for abatement of Escherichia coli at a treatment location as recited in claim 1, wherein said delivering said humic composition step comprises applying said humic composition as a topical treatment.

3. The method for abatement of Escherichia coli at a treatment location as recited in claim 2, said method for abatement further comprising:
placing said humic composition in a liquid solution; and
wherein said delivering said humic composition step further comprises applying said liquid solution to said treatment location.

4. The method for abatement of Escherichia coli at a treatment location as recited in claim 3, wherein said applying said liquid solution step comprises spraying said treatment location with said liquid solution.

5. The method for abatement of Escherichia coli at a treatment location as recited in claim 3, wherein said applying said liquid solution step comprises washing said treatment location with said liquid solution.

6. The method for abatement of Escherichia coli at a treatment location as recited in claim 3, wherein said applying said liquid solution step comprises dipping said treatment location in said liquid solution.

7. The method for abatement of Escherichia coli at a treatment location as recited in claim 3, wherein said applying said liquid solution comprises introducing said liquid solution to irrigation water.

8. The method for abatement of Escherichia coli at a treatment location as recited in claim 3, wherein said placing said humic composition in a liquid solution step comprises mixing said humic composition in water.

9. The method for abatement of Escherichia coli at a treatment location as recited in claim 2, said method for abatement further comprising:
mixing said humic composition with a personal care product; and
wherein said delivering said humic composition step further comprises applying said personal care product to said treatment location.

10. The method for abatement of Escherichia coli at a treatment location as recited in claim 1, wherein said delivering said humic composition step comprises releasing said humic composition as an enteral treatment.

11. The method for abatement of Escherichia coli at a treatment location as recited in claim 10, said method for abatement further comprising:
mixing said humic composition into an animal foodstuff; and
wherein said delivering said humic composition step further comprises feeding said animal foodstuff to an animal.

12. The method for abatement of Escherichia coli at a treatment location as recited in claim 10, said method for abatement further comprising:
combining said humic composition with a complex carbohydrate; and
wherein said delivering said humic composition step further comprises feeding said complex carbohydrate to an animal.

13. The method for abatement of Escherichia coli at a treatment location as recited in claim 12, wherein said complex carbohydrate comprises aloe.

14. The method for abatement of Escherichia coli at a treatment location as recited in claim 10, said method for abatement further comprising:
suspending said humic composition in a rectal suppository; and
wherein said delivering said humic composition step further comprises administering said rectal suppository to an animal.

15. The method for abatement of Escherichia coli at a treatment location as recited in claim 1, wherein said humic composition comprises at least 97.5% humic acid

16. The method for abatement of Escherichia coli at a treatment location as recited in claim 15, wherein said delivering said humic composition step comprises applying said humic composition as a topical treatment.

17. The method for abatement of Escherichia coli at a treatment location as recited in claim 15, wherein said delivering said humic composition step comprises releasing said humic composition as an enteral treatment.

18. The method for abatement of Escherichia coli at a treatment location as recited in claim 1, wherein said humic composition comprises at least 4.5% fulvic acid.

19. The method for abatement of Escherichia coli at a treatment location as recited in claim 18, wherein said delivering said humic composition step comprises applying said humic composition as a topical treatment.

20. The method for abatement of Escherichia coli at a treatment location as recited in claim 18, wherein said delivering said humic composition step comprises releasing said humic composition as an enteral treatment.
